(19) United States
(12) Patent Application Publication
(10) Pub. No.: US 2026/0184602 A1
(43) Pub. Date: Jul. 2, 2026

(54) WATER QUALITY pH DETECTION METHOD, WATER TREATMENT EQUIPMENT AND STORAGE MEDIUM

(71) Applicant: Guangdong Lizi Technology Co., Ltd., Foshan (CN)

(72) Inventors: Lin Shi, Foshan (CN); Xing Zhan, Foshan (CN); Guoxiang Chen, Foshan (CN); Yutai Huang, Foshan (CN); Wenbin Chen, Foshan (CN); Jiawei Chen, Foshan (CN); Yijie Hao, Foshan (CN); Mingyao Li, Foshan (CN); Jianbo Huang, Foshan (CN); Wei Luo, Foshan (CN); Erqiang Duan, Foshan (CN); Longbiao He, Foshan (CN)

(21) Appl. No.: 19/026,536

(22) Filed: Jan. 17, 2025

(30) Foreign Application Priority Data
Dec. 27, 2024 (CN) 202411944015.3
Dec. 27, 2024 (CN) 202411944017.2

(51) Int. Cl. C02F 1/00; C02F 1/66; G01N 33/18

(52) U.S. Cl. CPC C02F 1/008; C02F 1/66; G01N 33/18; C02F 2209/003; C02F 2209/006; C02F 2209/02; C02F 2209/06; C02F 2209/40; C02F 2209/44

(57) ABSTRACT

The present disclosure provides a water quality pH detection method, water treatment equipment, and a storage medium. The method acquires a first dissolved substance content value upstream of an alkaline filter material and a second dissolved substance content value downstream of the alkaline filter material in the water treatment equipment, providing a direct basis for subsequent pH value calculations. Based on the first and the second dissolved substance content values, a target difference value in dissolved substance content is calculated. A pH value of water is calculated and obtained based on the calculated target difference value in dissolved substance content, improving an accuracy of water quality detection. The present disclosure avoids a use of expensive pH meters and reduces a cost of water quality detection.

### CROSS REFERENCE TO RELATED APPLICATIONS

The present application claims the benefit of Chinese Patent Application Nos. 202411944017.2 and 202411944015.3 filed on Dec. 27, 2024, the contents of which are incorporated herein by reference in their entirety.

### FIELD

The present disclosure relates to a technical field of water purification, specifically a water quality pH detection method, water treatment equipment, and a storage medium.

### BACKGROUND

Adding alkaline filter materials to water treatment equipment not only enriches the water with beneficial minerals for the human body, but also significantly changes the pH value of water, making it closer to the ideal drinking water pH range, thereby enhancing the taste and nutritional value of the water. Therefore, accurate detection of the pH value of mineralized mineral water after treatment is key to ensuring water quality safety, improving user experience, and maintaining stable equipment operation.

In existing water treatment technologies, detecting the pH value of water usually relies on professional pH meters. However, configuring and using a pH meter will increase the overall cost of the equipment, and the pH meter is susceptible to environmental factors such as temperature, humidity, etc., during long-term use, which may cause deviations in the measurement results of the pH meter, thereby affecting the accuracy of the pH value detection in water.

### SUMMARY

In view of this, a water quality pH detection method, water treatment equipment, and a storage medium are provided, aiming to solve the technical problems of high cost and low accuracy in detecting water quality pH with existing water treatment equipment.

A first aspect of the present disclosure provides a water quality pH detection method, including:
acquiring a first dissolved substance content value upstream of an alkaline filter material and a second dissolved substance content value downstream of the alkaline filter material in water treatment equipment;
calculating a target difference value in dissolved substance content based on the first and the second dissolved substance content values; and
calculating and obtaining a pH value of water based on the calculated target difference value in dissolved substance content.

A second aspect of the present disclosure provides water treatment equipment, including at least one processor; a memory storing computer programs, wherein the at least one processor, when executing the computer programs, implements the following steps:
acquiring a first dissolved substance content value upstream of an alkaline filter material and a second dissolved substance content value downstream of the alkaline filter material in the water treatment equipment;
calculating a target difference value in dissolved substance content based on the first and the second dissolved substance content values; and
calculating and obtaining a pH value of water based on the calculated target difference value in dissolved substance content.

A third aspect of the present disclosure provides a non-transitory storage medium having stored thereon instructions that, when executed by a processor of water treatment equipment, causes the processor to perform a water quality pH detection method, the method including:
acquiring a first dissolved substance content value upstream of an alkaline filter material and a second dissolved substance content value downstream of the alkaline filter material in water treatment equipment;
calculating a target difference value in dissolved substance content based on the first and the second dissolved substance content values; and
calculating and obtaining a pH value of water based on the calculated target difference value in dissolved substance content.

The present disclosure directly obtains the first dissolved substance content value upstream and the second dissolved substance content value downstream of the alkaline filter material in the water treatment equipment, providing a direct basis for subsequent pH value calculations. Based on the second dissolved substance content value and the first dissolved substance content value, the target difference value in dissolved substance content is calculated. The target difference value in dissolved substance content represents changes in dissolved substance content of during a water treatment process and can be used to detect changes in water quality. Finally, based on the calculated target difference value in dissolved substance content, the pH value of water is calculated, greatly improving an accuracy of water quality detection. In addition, the present disclosure avoids a use of expensive pH meters and reduces a cost of water quality detection.

### BRIEF DESCRIPTION OF THE DRAWINGS

To describe the technical solutions in the embodiments of the present disclosure or in the related art more clearly, the following briefly introduces the accompanying drawings for describing the embodiments or the related art. Apparently, the accompanying drawings in the following description show merely some of the embodiments of the present disclosure, and a person of ordinary skill in the art can still derive other drawings from the accompanying drawings without creative efforts.

FIG. 1 shows a schematic flow chart of a water quality pH detection method provided by an embodiment of the present disclosure.

FIG. 2 shows a schematic diagram of TDS detection upstream and downstream of an alkaline filter material provided by an embodiment of the present disclosure.

FIG. 3 shows a schematic flow chart of a water quality pH detection method provided by another embodiment of the present disclosure.

FIG. 4 shows a schematic diagram of TDS detection upstream and downstream of an alkaline filter material provided by another embodiment of the present disclosure.

FIG. 5 shows a schematic structural diagram of water treatment equipment provided by an embodiment of the present disclosure.

### DETAILED DESCRIPTION

The technical solutions in the embodiments of the present disclosure are clearly and completely described below with reference to the accompanying drawings in the embodiments of the present disclosure. Apparently, the described embodiments are merely some rather than all of the embodiments of the present disclosure. All other embodiments obtained by a person of ordinary skill in the art based on the embodiments of the present disclosure without making creative efforts shall fall within the protection scope of the present disclosure.

A water quality pH detection method provided in the embodiment of the present disclosure is mainly applied to water treatment equipment in household or office places to detect a pH value of water of water outputted by the water treatment equipment.

The water treatment equipment is a device used to improve water quality, remove impurities from water, or adjust water quality, such as a water purifier or filtration system, etc. The water treatment equipment in the embodiment of the present disclosure is a water purifier system with mineralization function. The water purifier system with mineralization function refers to a type of water purification equipment that can deeply purify tap water and add mineral elements necessary for the human body to make the water quality reach standards similar to mineral water. After removing harmful substances, the water purifier system with mineralization function re integrates minerals such as calcium, magnesium, iron, and zinc, which are essential for the human body, into the water by adding mineral rich filter materials. These minerals exist in an ionic state, making them easier for the human body to absorb.

The water treatment equipment can include a filter element component, a filter, a reverse osmosis device, and so on. In an embodiment of the present disclosure, the filter element component can include one or more alkaline filter materials. The alkaline filter material is a type of filter element or material used in water treatment, whose primary function is to convert acidic water quality into alkaline water quality. This is typically achieved by reducing the acidity of water through negative ions, thereby increasing the alkalinity of water.

One alkaline filter material corresponds to one alkaline water path. When more alkaline filter materials are employed, water channels between them are usually arranged in a parallel configuration to ensure that the water flows can pass evenly through each filter material.

In the embodiment of the present disclosure, the alkaline filter material contains alkaline substances. For multiple alkaline filter materials, types and dissolution rates of the alkaline substances contained inside different alkaline filter materials are different. Therefore, alkaline strengths of different alkaline filter materials vary, achieving different alkaline treatment effects.

Next, taking two alkaline filter materials as examples, the present disclosure will illustrate an interaction between alkaline filter materials and their effectiveness in water treatment.

For example, in a filter element component, a first alkaline filter material contains weakly acidic ores, while a second alkaline filter material contains ores rich in calcium and magnesium. When water flows through the first alkaline filter material first, the acidic substances released from the first alkaline filter material can effectively promote the release of calcium and magnesium elements from the subsequent second alkaline filter material. This promoting effect not only improves mineralization efficiency, but also enables water quality to be rich in beneficial minerals for the human body after passing through the entire filter element component.

Again, in another example of a filter element component, a first alkaline filter material contains weakly alkaline ores, while a second alkaline filter material contains ores containing zinc and copper. When water flows through the first alkaline filter material first, the alkaline substances released from the first alkaline filter material will inhibit the release of zinc and copper elements from the second alkaline filter material. This inhibitory relationship can control a content of certain minerals in water.

In another example, in a filter element component, a first alkaline filter material contains different amounts of alkaline ore, while a second alkaline filter material contains different amounts of calcium and magnesium ore. By adjusting a flow rate of a water channel between the first alkaline filter material and the second alkaline filter material, a release concentration of different mineralized elements can be flexibly adjusted to satisfy specific water quality needs of different users.

In a design of the filter element component, an arrangement and a combination of different filter materials are also crucial. For example, a combination of surface strongly alkaline materials (such as brucite) and inner weakly alkaline materials (such as calcite) is adopted. This design allows the filter element to quickly release strong alkaline substances in an initial stage, and over time, the weak alkaline material in the inner layer gradually comes into play, ensuring that the filter element maintains a moderate alkaline strength throughout its entire service life.

By designing and combining different types of alkaline filter materials along with their waterway configurations, precise mineralization treatment of water quality can be achieved. This not only helps to improve safety and health of water quality, but also meets the personalized needs of different users for water quality taste and nutritional value.

According to relevant regulations of the “Hygienic Standards for Drinking Water”, a normal pH range of drinking water is 6.5-8.5. This is because low pH can easily corrode metal water pipelines, while high pH can cause calcium and magnesium ions in the water to precipitate and form scale, which can adversely affect human health.

Given numerous benefits of alkaline water for human health and clear regulations on the pH value of water in the “Hygienic Standards for Drinking Water” , it is particularly important to accurately detect the pH value of water output from water purifier system with mineralization function. This not only concerns the safety and health of users'drinking water, but also directly affects the stable operation of equipment and user experience.

In order to achieve this objective, embodiments of the present disclosure provide a water quality pH detection method applied to water treatment equipment in household or office places. This method aims to ensure that the mineralized water is safe and meets health standards when it reaches a user end by detecting the pH value of water in real time.

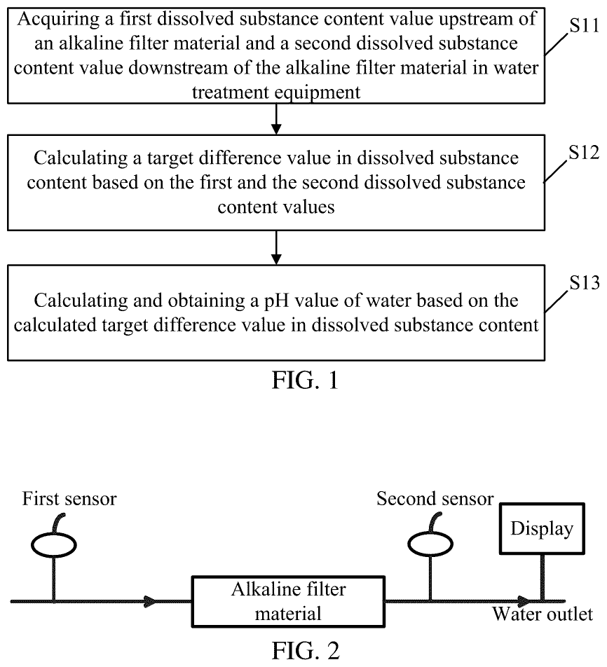

As shown in FIG. 1, a schematic flow chart of a water quality pH detection method provided by an embodiment of the present disclosure. The water quality pH detection method applicable in water treatment equipment can include the following steps.

In block S11, acquiring a first dissolved substance content value upstream of an alkaline filter material and a second dissolved substance content value downstream of the alkaline filter material in water treatment equipment.

In a field of water treatment, the dissolved substance content value is usually used to measure a total amount of dissolved substances in water, which can include salts, minerals, organic matter, etc. By detecting the dissolved substance content value, a quality of water and an effectiveness of water treatment equipment can be evaluated. In the embodiment of the present disclosure, in order to more accurately detect the pH of water quality in water treatment equipment, the dissolved substance content value includes a total dissolved solids (TDS) value or an electrical conductivity.

The total dissolved solids (TDS) value refers to a total amount of solids dissolved in water, including content of both inorganic and organic substances. A measurement unit is milligrams per liter (1 mg/L=1 ppm), indicating how many milligrams of total solids are dissolved in one liter of water. A higher the TDS value signifies that there are more dissolved substances in the water. Generally, the electrical conductivity value can be used to roughly estimate the salt content in a solution. The higher the electrical conductivity, the higher the salt content, and the higher the TDS value. A TDS sensor is an instrument that measures the electrical conductivity of water to reflect a degree of mineralization of the water quality. A TDS sensor is an instrument that measures the electrical conductivity of water to reflect the degree.

The electrical conductivity is an indicator of ion concentration in water, which reflects an ability of dissolved substances in water to conduct electricity. Dissolved substances in water can form ions, which can transfer current when moving in water. Therefore, electrical conductivity can be used to evaluate the ion concentration in water. The higher the electrical conductivity, the higher the ion concentration in the water, potentially containing more dissolved substances. In a process of water treatment, electrical conductivity can be used to detect changes in water quality and evaluate the effectiveness of water treatment equipment.

As shown in FIG. 2, a first sensor is installed upstream of the alkaline filter material. The first sensor is used to detect a dissolved substance content value upstream of the alkaline filter material (i.e., the first dissolved substance content value). The first dissolved substance content value is a dissolved substance content value before the water flows through the alkaline filter material, which is a dissolved substance content value of the raw water. Similarly, a second sensor is installed downstream of the alkaline filter material. The second sensor is used to detect a dissolved substance content value downstream of the alkaline filter material (i.e., the second dissolved substance content value). The second dissolved substance content value is a dissolved substance content value after the water flows through the alkaline filter material.

In block S12, calculating a target difference value in dissolved substance content based on the first and the second dissolved substance content values.

To obtain the target difference value in dissolved substance content, the first dissolved substance content value can be subtracted from the second dissolved substance content value.

Assuming the first sensor is a first TDS sensor, which detects the first dissolved substance content value upstream of the alkaline filter material as TDS1, and the second sensor is a second TDS sensor, which detects the second dissolved substance content value downstream of the alkaline filter material as TDS2, then the target difference (ΔTDS) in dissolved substance content can be expressed as: ΔTDS=TDS2−TDS1. Assuming the first sensor is a first electrical conductivity sensor, which detects the first dissolved substance content value upstream of the alkaline filter material as electrical conductivity 1, and the second sensor is a second electrical conductivity sensor, which detects the second dissolved substance content value downstream of the alkaline filter material as electrical conductivity 2, then the target difference (Δ electrical conductivity) in dissolved substance content can be expressed as: Δ electrical conductivity=electrical conductivity 2−electrical conductivity 1.
The target difference value in dissolved substance content represents a change in dissolved substance content of water quality during a treatment process, which can be used to evaluate a performance of the water treatment equipment or detect changes in water quality.

It should be understood that in water purifier systems with mineralization function, the target difference value in dissolved substance content is usually not less than 0. The reason is that mineralized water purifiers incorporate filter materials rich in minerals (such as calcium, magnesium, zinc, and other ores) to integrate the mineral elements needed by the human body into the water. These minerals exist in the water in ion or dissolved forms, which increases the content of dissolved substance in the water. Even though purification treatment is carried out before mineralization to remove harmful substances and impurities from the water, the reduction mainly involves non-mineral dissolved solids. The minerals added during the mineralization process will compensate for or even exceed this reduction, resulting in an overall increase in the dissolved substance content value.

During a water treatment process, changes in water flow speed can affect the distribution, mixing, and transportation of dissolved substances in the water. Rapid water flow may agitate sediments, releasing more dissolved substances into the water, thereby affecting the measurement of dissolved substance content values. In addition, the measurement of dissolved substance content values usually depends on physical properties such as electrical conductivity, and changes in water flow speed can affect the measurement of these physical properties. For example, changes in water flow speed can affect a contact area and a contact time between electrodes and water bodies, thereby affecting the measurement of the electrical conductivity. Therefore, it is necessary to correct the dissolved substance content value based on water flow speed.

In an optional embodiment, the step of calculating a target difference value in dissolved substance content based on the first and the second dissolved substance content values, includes the following:
acquiring a first water flow speed upstream of the alkaline filter material and a second water flow speed downstream of the alkaline filter material;
correcting the first dissolved substance content value using the first water flow speed to obtain a first corrected dissolved substance content value;
correcting the second dissolved substance content value using the second water flow speed to obtain a second corrected dissolved substance content value;
obtaining the target difference value in dissolved substance content based on the first and the second corrected dissolved substance content values.

A first water flow speed sensor or flow meter is installed upstream of the alkaline filter material. The first water flow speed sensor or flow meter is used to detect a water flow speed upstream of the alkaline filter material (i.e., the first water flow speed). The first water flow speed is a speed before the water flows through the alkaline filter material, reflecting a flow state of the water before entering the filter material. Similarly, a second water flow speed sensor or flow meter is installed downstream of the alkaline filter material. The second water flow speed sensor or flow meter is used to detect a water flow speed downstream of the alkaline filter material (i.e., the second water flow speed). The second water flow speed is a speed after the water flows through the alkaline filter material, reflecting a flow state of the water after being treated by the filter material.

When the water flow speed increases, a scouring effect of the water flow on the sensor may be enhanced, resulting in a lower sensor readings; On the contrary, when the water flow speed decreases, the scouring effect of the water flow on the sensor weakens, and potentially resulting in higher sensor readings. Therefore, after obtaining the first water flow speed upstream and the second water flow speed downstream of the alkaline filter material, a correction model needs to be applied to correct the first dissolved substance content value based on the first water flow speed and the second dissolved substance content value based on the second water flow speed, to eliminate measurement errors in the dissolved substance content values upstream and downstream of the alkaline filter material caused by changes in water flow speed. The corrected values (i.e. the first corrected dissolved substance content value and the second corrected dissolved substance content value) can more accurately reflect changes in water quality during the treatment process.

After obtaining the first corrected dissolved substance content value and the second corrected dissolved substance content value, the target difference value in dissolved substance content is obtained based on a difference between the first corrected dissolved substance content value and the second corrected dissolved substance content value. The target difference value in dissolved substance content can more accurately reflect changes in water quality, thereby more accurately evaluating a performance of water treatment equipment and detecting changes in water quality.

The above optional embodiment, by setting water flow speed sensors to detect the water flow speed separately upstream and downstream of the alkaline filter material, can comprehensively reflect the changes in a water flow state during the treatment process, especially when there is a significant difference value in water flow speed between the upstream and downstream of the alkaline filter material. By using the water flow speed to correct the corresponding dissolved substance content values, the more accurate target difference value in dissolved substance content can be obtained, which can be used to evaluate the performance of water treatment equipment or detect changes in water quality.

In an optional embodiment, the step of correcting the first dissolved substance content value using the first water flow speed to obtain a first corrected dissolved substance content value, includes the following:
determining a change in water flow speed based on the first water flow speed and a first water flow speed of a previous cycle;
correcting the first dissolved substance content value based on the first water flow speed and the change in water flow speed through a preset first correction model, and obtaining the first corrected dissolved substance content value.

Wherein, the preset first correction model is represented as follows:

Y1_corrected=Y1* 1+ΔV/1*k1).

V1 is the first water flow speed. ΔV is the change in water flow speed, which is a difference between the current water flow speed (i.e., first water flow speed V1) and the first water flow speed value of the previous cycle. The change in water flow speed reflects a fluctuation of water flow speed over a period of time. K1 is a correction factor, which is a coefficient determined based on experiments or experience, used to adjust measurement errors in dissolved substance content value caused by changes in water flow speed. Y1 is the first dissolved substance content value. Y1_comrrected is the first corrected dissolved substance content value. Cycle refers to a collection period of the first water flow speed sensor.

ΔV/V1 represents a rate of change in water flow speed, which is a magnitude and trend of the change in water flow speed relative to the first water flow speed V1. It reflects how the water flow speed changes, whether it increases or decreases, and the magnitude of the change.

ΔV/V1*k1 represents an adjustment amount for the measurement error of dissolved substance content value caused by changes in water flow speed. It takes into account both the rate of change in water flow speed and the correction factor, and is used to correct the original dissolved substance content value.

Directly correcting based on water flow speed may not fully reflect the actual impact of water flow speed fluctuations on the measurement of dissolved substance content values. This is because the change in water flow speed is not only related to a current speed, but also closely related to the trend and magnitude of changes in speed. Therefore, correcting based on the change in water flow speed can more accurately reflect the impact of water flow speed fluctuations on the measurement of dissolved substance content values. By considering the trend and magnitude of changes in speed, the dissolved substance content values can be adjusted more accurately, thereby improving an accuracy of the measurement.

The above optional embodiment quantifies the impact of changes in water flow speed on the measurement of dissolved substance content values through the correction factor k1, the change in water flow speed ΔV and the first water flow speed. This makes the correction process more scientific and accurate. The first correction model can adapt to correction requirements under different water flow speeds. Whether the water flow speed increases or decreases, dynamic correction of the dissolved substance content values can be achieved through the correction factor and the changes in speed, thereby improving accuracy and reliability of the measurement.

In an optional embodiment, the step of correcting the second dissolved substance content value using the second water flow speed to obtain a second corrected dissolved substance content value, includes the following:
calculating a water residence time based on a volume of the alkaline filter material and the second water flow speed;
determining a mass transfer coefficient based on the second water flow speed;
correcting the second dissolved substance content value based on a preset reference water flow speed, the water residence time, the mass transfer coefficient, and the second water flow speed through a preset second correction model, and obtaining the second corrected dissolved substance content value.

Wherein, the preset second correction model is represented as follows:

Y2_corrected=Y2*(1−k2*t/(1+k2*t)*(V2/V0−1)).

V2 represents the second water flow speed. k2 represents the mass transfer coefficient, which is an empirical coefficient that reflects an efficiency of material exchange between the water flow and the filter material. The mass transfer coefficient is used to adjust measurement errors in TDS (Total Dissolved Solids) or electrical conductivity caused by changes in water flow speed. t stands for the water residence time, which is the time required for water to pass through the filter material and depends on a volume of the filter material and the water flow speed. V0 is the reference water flow speed, which is a standard or benchmark water flow speed for comparison with the current water flow speed. A selection of the reference water flow speed may be based on experimental data, industry standards, or empirical values. Y2 represents the second dissolved substance content value, while Y2_corrected stands for the second corrected dissolved substance content value.

The expression k2*t/(1+k2*t) is a function of the mass transfer coefficient k2 and the water residence time t, representing the measurement error in TDS (Total Dissolved Solids) or electrical conductivity due to the material exchange between the water flow and the filter material. As the water residence time t increases, the material exchange becomes more thorough, which may affect measurement results of the TDS or the electrical conductivity. The mass transfer coefficient k2 reflects the efficiency of this material exchange. Since k2 is a dimensionless coefficient, the expression k2*t/(1+k2*t) is also dimensionless.

V2/V0 represents a ratio between the current water flow speed V2 and the reference water flow speed V0, indicating an impact of changes in water flow speed on measurements of the TDS (Total Dissolved Solids) or the electrical conductivity. When the water flow speed changes, it may affect a contact area and contact time between the water flow and the filter material, thereby affecting the measurements results of the TDS or the electrical conductivity. Since V2 and V0 have the same dimension, the ratio V2/V0 is dimensionless as a whole.

When the water flow speed V2 changes, the water residence time t and the mass transfer coefficient k2 are also affected. These changes will cause changes in the correction factor, which in turn affect the corrected TDS (Total Dissolved Solids) or the corrected electrical conductivity values. The model adjusts the correction factor to reflect the impact of these changes on the measurements of the TDS or the electrical conductivity.

Changes in water flow speed affect the contact area and contact time between the water flow and the filter material, which in turn impact the material exchange and the measurement results of the TDS (Total Dissolved Solids) or the electrical conductivity. The water residence time determines a degree of material exchange between the water flow and the filter material. The longer the residence time, the more thorough the material exchange. The mass transfer coefficient reflects the efficiency of material exchange between the water flow and the filter material. Therefore, the second correction model achieves correction of the second dissolved substance content by comprehensively considering the impact of the water flow speed, the water residence time, the mass transfer coefficient, and the reference water flow speed on the measurement of dissolved substance content. This allows for a more accurate reflection of actual water quality conditions and improves the accuracy of dissolved substance content.

In block S13, calculating and obtaining a pH value of water based on the calculated target difference value in dissolved substance content.

In order to obtain the pH value of water based on the calculated target difference value in dissolved substance content, it is necessary to collect water quality sample data from various water sources and conditions in advance. Based on the water quality sample data, a potential correlation or functional relationship between difference values in dissolved substance content and pH values can be fitted. Subsequently, using the fitted correlation or functional relationship, the pH value of water can be calculated based on the calculated target difference value in dissolved substance content.

The water quality sample data can include difference values in dissolved substance content and corresponding pH values. Collecting the water quality sample data from different water sources and conditions is to ensure that the functional relationship fitted based on the water quality sample data has sufficient generalization ability. Water quality conditions are dynamically changing and impacted by various factors. By collecting diversified water quality sample data, it can ensure that the model can learn complex relationships between different water quality characteristics, in order to better adapt to trends of water quality parameters changing over time and space, and thus providing more reliable prediction results.

The collected water quality sample data can be preprocessed, including removing outliers and smoothing data, etc., to improve data quality.

An appropriate data fitting algorithm (such as a linear regression, a polynomial regression, a machine learning algorithm, etc.) can be used to fit the functional relationship between difference values in dissolved substance content and pH values, to obtain a mathematical function that best describes the relationship between the two. For example, by linearly fitting TDS difference values and pH values, a following relationship equation can be obtained: f(ΔTDS)=a+b*ΔTDS, wherein a is a source term constant, and b is an amplitude index, both of which are related to alkaline properties of the mineralized filter material and are measured experimentally.

In water quality detection, there is often significant uncertainty in predicting the pH value of water directly through the TDS differences. Especially when using alkaline filter materials for water quality treatment, types and properties of the filter materials can significantly affect chemical balance of water body. Different types of the alkaline filter materials have different chemical compositions, pore structures, and surface properties. These differences can lead to different effects of the alkaline filter materials during water treatment, further affecting the relationship between the TDS differences and the water pH values.

In an optional embodiment, the step of calculating and obtaining a pH value of water based on the calculated target difference value in dissolved substance content, includes the following:
determining a type of the alkaline filter material;
selecting a first target water quality pH detection model from a plurality of preset first water quality pH detection models based on the type; and
obtaining the pH value of water through the first target water quality pH detection model based on the calculated target difference value in dissolved substance content.

The type of the alkaline filter material used can be determined by consulting a product manual. Based on the determined type of the alkaline filter material, the model that matches the type of the alkaline filter material is selected from the plurality of preset first water quality pH detection models stored locally. The target difference value in dissolved substance content is then input into the selected model. The selected model calculates the pH value of water based on the input target difference value in dissolved substance content.

In order to establish a model that accurately reflects the relationship between types of the alkaline filter material, difference values in dissolved substance content, and pH values, a large amount of experimental data can be collected. These data include the dissolved substance content values and pH values of water quality samples before and after treatment with different types of alkaline filter materials. For each type of alkaline filter material, a function model is established by simulating the relationship between the difference values in dissolved substance content before and after treatment with the alkaline filter materials and the pH values of water quality, resulting in the first water quality pH detection model.

The above optional embodiment considers the type of the alkaline filter material and selects a suitable model from the plurality of preset first water quality pH detection models based on the type of alkaline filter material. This allows for a more accurate calculation of the pH value of water based on the calculated target difference value in dissolved substance content, thereby improving the accuracy of the pH value detection in water.

During a process of using water treatment equipment to improve water quality, water bodies in different locations may be affected by different environmental factors, such as temperature, light exposure, geological conditions, and human activities. These factors may all lead to changes in dissolved substance content of the water bodies, thereby affecting the relationship between the difference values in dissolved substance content and the pH values. Therefore, considering the location of the water treatment equipment as an important variable when building the model can help to more accurately predict the pH value of water.

In an optional embodiment, the step of calculating and obtaining a pH value of water based on the calculated target difference value in dissolved substance content, includes the following:
determining a location of the water treatment equipment;
selecting a second target water quality pH detection model from a plurality of preset second water quality pH detection models based on the location; and
obtaining the pH value of water through the second target water quality pH detection model based on the calculated target difference value in dissolved substance content.

GIS technology can be used to determine the precise location of water treatment equipment through map positioning and data analysis. Based on the determined location of the water treatment equipment, the model that matches the location of the water treatment equipment is selected from the plurality of preset second water quality pH detection models stored locally. The target difference value in dissolved substance content is then input into the selected model. The selected model calculates the pH value of water based on the input target difference value in dissolved substance content.

In order to establish a model that accurately reflects the relationship between locations of water treatment equipment, difference values in dissolved substance content, and pH values, a large amount of experimental data can be collected. These data include the dissolved substance content values and pH values of water quality samples before and after treatment with the alkaline filter materials at different locations. For each location of water treatment equipment, a function model is established by simulating the relationship between the difference values in dissolved substance content before and after treatment with alkaline filter materials and the pH values of water quality, resulting in the second water quality pH detection model.

The above optional embodiment considers the location of the water treatment equipment and selects a suitable model from the plurality of preset second water quality pH detection models based on the location of the water treatment equipment. This allows for a more accurate calculation of the pH value of water based on the calculated target difference value in dissolved substance content, thereby improving the accuracy of the pH value detection in water.

In an optional embodiment, the step of calculating and obtaining a pH value of water based on the calculated target difference value in dissolved substance content, includes the following:
determining a type of the alkaline filter material and a location of the water treatment equipment;
selecting a third target water quality pH detection model from a plurality of preset third water quality pH detection models based on the type and the location; and
obtaining the pH value of water through the third target water quality pH detection model based on the calculated target difference value in dissolved substance content.

The third water quality pH detection model is a function model established by simulating the relationship between difference values in dissolved substance content before and after filtration, and pH values of water quality, for different types of alkaline filter material and water quality at various locations.

The above optional embodiment selects a model that matches the type of the alkaline filter material and the position of the water treatment equipment from the plurality of preset third water quality pH detection models stored locally, based on the determined location of the water treatment equipment and the type of alkaline filter material. This allows for a more accurate calculation of the pH value of water based on the calculated target difference value in dissolved substance content, thereby improving the accuracy of the pH value detection in water.

In an optional embodiment, the method further includes:
displaying the pH value of water using a preset display mode.

After the pH value detection in water, the pH value of water is saved in real-time for subsequent analysis of water quality and water state. At the same time, the pH value of water is displayed on a display interface at an end of the waterway, allowing people to intuitively understand whether the water quality in the water treatment equipment is more suitable for people to drink.

The preset display mode can include one or a combinations of various display types such as a color-coding, a graphical representation, a numerical display.

The color-coding refers to a display of different colors to display based on range of pH values of water quality. For example, green is used to indicate good water quality (pH value close to neutral), yellow is used to indicate average water quality (pH value slightly deviating from neutral), and red is used to indicate poor water quality (pH value significantly deviating from neutral). This color-coding display can visually reflect the water quality, facilitating users to make quick judgments.

The graphical representation refers to a display of trends in pH values of water quality through various chart formats such as bar charts and line graphs. These charts can clearly show how pH values of water quality change over time, space, and other factors, helping users identify potential problems or patterns.

The numerical display refers to a direct display of pH values of water quality on a screen. This can provide precise data information, facilitating users to conduct quantitative analysis and comparisons.

Additionally, the digital display can also be combined with the color coding, the graphical representation, color bars or color lights to form a more comprehensive way of information display.

In practical applications, the preset display mode can be flexibly adjusted according to user needs, application scenarios, and other factors.

The above optional embodiment, which visualizes the pH value of water after detecting, can improve user experience and enhance data readability.

In an optional embodiment, the method further includes:
comparing the pH value of water with a preset pH threshold range; and
triggering an alarm according to a preset alarm mode, when the pH value of water exceeds the preset pH threshold range.

The preset pH threshold range for water quality is usually determined based on water quality standards, industry standards, or requirements of specific application scenarios. Optionally, the preset pH threshold range for water quality can be set between 6.5 and 8.5.

The real-time detected pH value of water is compared with the preset pH threshold range. If the pH value of water falls within the preset pH threshold range, it indicates that the water quality is within a normal or ideal range of acidity and alkalinity, and the water quality meets the standards. If the pH value of water exceeds the preset pH threshold range, for example, with a pH less than 6.5 or greater than 8.5, it indicates that the water quality does not meet the standards.

When the pH value of water discharged from the water treatment equipment exceeds the preset pH threshold range, sometimes users notice it immediately. Therefore, the water treatment equipment in the embodiment of the present disclosure is equipped with an audible and visual alarm module. The audible and visual alarm module is electrically connected to a central processing module and is controlled and executed uniformly by the central processing module. The alarm module can use sound and/or light to give alarm prompts. This prompt method can attract users'attention and take timely measures to address water quality problems. The alarm content can include real-time data on water quality pH value, specific situations exceeding the threshold (such as exceeding an upper or a lower limit), possible hazards, and recommended response measures. These pieces of information help users or relevant personnel quickly understand water quality issues and take corresponding handling measures.

Additionally, SMS notifications, email alerts, and other methods can also be employed for alarm prompts. Different alarm methods can be selected based on different application scenarios and user needs.

The above optional embodiment compares the pH value of water with the preset pH threshold range, and takes corresponding alarm measures based on the comparison results, which not only improves an automation and an efficiency of water quality detection, but also ensures that timely alerts can be issued when water quality does not meet standards, thereby effectively preventing potential environmental and health risks.

The present disclosure directly obtains the first dissolved substance content value upstream and the second dissolved substance content value downstream of the alkaline filter material in the water treatment equipment, providing a direct basis for subsequent pH value calculations. Based on the second dissolved substance content value and the first dissolved substance content value, the target difference value in dissolved substance content is calculated. The target difference value in dissolved substance content represents changes in dissolved substance content of the water during the treatment process and can be used to detect changes in water quality. Finally, based on the calculated target difference value in dissolved substance content, the pH value of water is calculated, greatly improving an accuracy of water quality detection. In addition, the present disclosure avoids a use of expensive pH meters and reduces a cost of water quality detection.

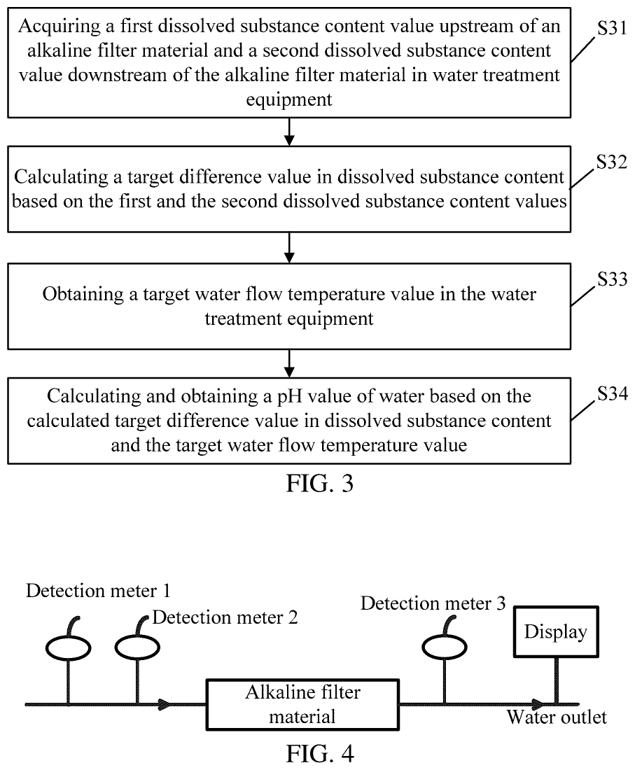

FIG. 3 shows a schematic flow chart of a water quality pH detection method provided by another embodiment of the present disclosure. The water quality pH detection method applicable in water treatment equipment can include the following steps.

In block S31, acquiring a first dissolved substance content value upstream of an alkaline filter material and a second dissolved substance content value downstream of the alkaline filter material in water treatment equipment.

A first sensor can be installed at an appropriate position in the water treatment equipment, for example, as shown in FIG. 4, a detection meter 2 can be installed upstream of a alkaline filter material in the water treatment equipment. The first sensor is used to detect a dissolved substance content value upstream of the alkaline filter material (i.e., the first dissolved substance content value). The first dissolved substance content value is a dissolved substance content value before the water flows through the alkaline filter material, which is a dissolved substance content value of the raw water. Similarly, a second sensor can be installed at an appropriate position in the water treatment equipment, for example, as shown in FIG. 4, a detection meter 3 can be installed downstream of a alkaline filter material in the water treatment equipment. The second sensor is used to detect a dissolved substance content value downstream of the alkaline filter material (i.e., the second dissolved substance content value). The second dissolved substance content value is a dissolved substance content value after the water flows through the alkaline filter material.

In block S32, calculating a target difference value in dissolved substance content based on the first and the second dissolved substance content values.

Assuming the first sensor is a first TDS sensor, which detects the first dissolved substance content value upstream of the alkaline filter material as TDS1, and the second sensor is a second TDS sensor, which detects the second dissolved substance content value downstream of the alkaline filter material as TDS2, then the target difference (ΔTDS) in dissolved substance content can be expressed as: ΔTDS=TDS2−TDS1. Assuming the first sensor is a first electrical conductivity sensor, which detects the first dissolved substance content value upstream of the alkaline filter material as electrical conductivity 1, and the second sensor is a second electrical conductivity sensor, which detects the second dissolved substance content value downstream of the alkaline filter material as electrical conductivity 2, then the target difference (Δ electrical conductivity) in dissolved substance content can be expressed as: Δ electrical conductivity=electrical conductivity 2−electrical conductivity 1.

The target difference value in dissolved substance content represents a change in dissolved substance content of water quality during a treatment process, which can be used to evaluate a performance of the water treatment equipment or detect changes in water quality.

In block S33, obtaining a target water flow temperature value in the water treatment equipment.

A water flow temperature value in the water treatment equipment is one of the important factors affecting a pH value of water. At different temperatures, a degree of ionization of water varies, which affects the pH value of water output from water treatment equipment.

A temperature sensor can be installed at an appropriate position in the water treatment equipment, for example, as shown in FIG. 4, a detection meter 1 can be installed upstream of the alkaline filter material in the water treatment equipment. The temperature sensor is used to detect and record the water flow temperature value in real time.

In practical applications, the water flow temperature value in the water treatment equipment may vary due to various factors. If temperature correction is not performed, the pH value of water measured at different temperatures may have significant deviations. Therefore, in order to ensure an accuracy of measuring the pH value of water, it is necessary to correct the real-time water flow temperature value in the water treatment equipment to obtain the target water flow temperature value.

In an optional embodiment, the step of obtaining a target water flow temperature value in the water treatment equipment, includes the following:
acquiring a real-time water flow temperature value in the water treatment equipment;
comparing the real-time water flow temperature value with a preset reference temperature range;
when the real-time water flow temperature value is within the preset reference temperature range, correcting the real-time water flow temperature value using a preset temperature correction coefficient to obtain the target water flow temperature value;
when the real-time water flow temperature value is not within the preset reference temperature range, adjusting the preset temperature correction coefficient, and correcting the real-time water flow temperature value using the adjusted temperature correction coefficient to obtain the target water flow temperature value.

The preset reference temperature range is set based on factors such as experience, performance of the water treatment equipment, material and characteristics of filter materials, etc., representing a most suitable water flow temperature range for measuring the dissolved substance content. That is to say, accurate and reliable results can be obtained by measuring the dissolved substance content within the reference temperature range.

The real-time detected water flow temperature value is compared with the preset reference temperature range, and based on the comparison result, a decision is made on whether to adjust the temperature correction coefficient. When the comparison result indicates that the real-time water flow temperature value is within the preset reference temperature range, the temperature correction coefficient is not adjusted. When the comparison result indicates that the real-time water flow temperature value is not within the preset reference temperature range, the temperature correction coefficient is adjusted.

When the real-time water flow temperature value is within the preset reference temperature range, it indicates that the real-time water flow temperature is within a considered normal or ideal operating temperature range. Within this range, the preset temperature correction coefficient (which can be a normal number based on previous experiments or experience, or a preset temperature correction coefficient of 0) is considered sufficiently accurate and reliable for directly correcting the real-time water flow temperature value to obtain the target water flow temperature value. At this point, the impact of temperature on pH measurement can be considered controllable and known, so there is no need to make additional adjustments to the temperature correction coefficient. However, when the real-time water flow temperature value is not within the preset reference temperature range, it indicates that the real-time water flow temperature has deviated from the normal or ideal operating temperature range. In this case, the preset temperature correction coefficient may no longer be accurate or reliable, as temperature changes may have caused significant variations in electrode potential, solution properties, or other factors that affect the pH measurement. Therefore, adjustments to the preset temperature correction coefficient are needed to reflect the actual measurement conditions at the current temperature. The adjusted temperature correction coefficient will be used to correct the real-time water flow temperature value, resulting in a more accurate target water flow temperature value.

The aforementioned optional embodiment can significantly improve the accuracy of temperature measurement by comparing the real-time water flow temperature value with the preset reference temperature range and selecting whether to adjust the temperature correction coefficient based on the comparison result, thereby ensuring that the final obtained pH value obtained is based on accurate temperature data. When the real-time water flow temperature value is not within the preset reference temperature range, the temperature correction coefficient can be automatically adjusted. This adaptive capability enables the system to maintain the accuracy of measurement results under different temperature conditions, enhancing the robustness and adaptability of the system. Furthermore, through the automated temperature correction process, the operation process of pH measurement has been simplified. Operators do not need to manually adjust the temperature or the temperature correction coefficient, as these steps are automatically completed, thereby improving work efficiency.

In an optional embodiment, the step of adjusting the preset temperature correction coefficient, includes the following:
obtaining a temperature deviation degree based on the real-time water flow temperature value and the preset reference temperature range;
adjusting the preset temperature correction coefficient according to the temperature deviation degree.

By calculating a difference or a ratio between the real-time water flow temperature value and a central value (or a specific value, such as an upper limit or a lower limit) of the preset reference temperature value, a numerical value representing the temperature deviation degree can be obtained. The temperature deviation degree reflects a magnitude of the difference between the current real-time water flow temperature value and an optimal measurement temperature.

If the temperature deviation degree is small, it indicates that the difference between the real-time water flow temperature value and the central value (or specific value) of the preset reference temperature range is not significant, which means that the current temperature are close to the optimal measurement temperature. In this case, fine-tuning the preset temperature correction coefficient is sufficient. A magnitude of the adjustment can be relatively small because the temperature deviation degree is not large, and the impact on pH measurement is also relatively minor. By fine-tuning the preset temperature correction coefficient, the impact of temperature on the measurement results can be further reduced, the accuracy of the measurement can be improved.

If the temperature deviation degree is large, it indicates that the difference between the real-time water flow temperature value and the central value (or specific value) of the preset reference temperature range is significant, which means that the current temperature are far from the optimal measurement temperature. In this case, a larger adjustment to the preset temperature correction coefficient is required. Due to the large temperature deviation degree, the impact on pH measurement will also be relatively large. Therefore, a larger adjustment to the preset temperature correction coefficient is needed to compensate for the impact of temperature on the measurement results.

The aforementioned optional embodiment can quantify impact of temperature on the measurement by calculating the temperature deviation degree, thereby providing a reliable basis for subsequent adjustments to the preset temperature correction coefficient. By appropriately adjusting the preset temperature correction coefficient based on the temperature deviation degree, the accuracy of pH measurement can be further improved. For cases with a small temperature deviation degree, fine adjustments are sufficient; for cases with a large temperature deviation degree, large adjustments are required. The embodiment of the present disclosure adjusts the temperature correction coefficient based on the temperature deviation degree, enabling the system to maintain the accuracy of measurement results under different temperature conditions, enhancing the adaptability and robustness of the system.

In an optional embodiment, the step of adjusting the preset temperature correction coefficient according to the temperature deviation degree, includes the following:
obtaining a temperature deviation severity based on the temperature deviation degree and a preset temperature deviation degree threshold;
determining a water flow speed value of the alkaline filter material;
obtaining a correction adjustment coefficient based on the water flow speed value and the temperature deviation severity;
adjusting the preset temperature correction coefficient based on the correction adjustment coefficient.

Water flow speed value refers to a displacement of water flow in a unit of time, typically measured in meters per second (m/s). A first water flow speed sensor or flowmeter can be installed upstream of the alkaline filter material. The first water flow speed sensor or flowmeter is configured to detect a water flow speed value upstream of the alkaline filter material (i.e., the first water flow speed value). The first water flow speed value represents a speed value of the water before passing through the alkaline filter material, reflecting a flow state of the water entering the alkaline filter material. Similarly, a second water flow speed sensor or flowmeter can be installed downstream of the alkaline filter material. The second water flow speed sensor or flowmeter is configured to detect a water flow speed value downstream of the alkaline filter material (i.e., the second water flow speed value). The second water flow speed value represents a speed value of the water after passing through the alkaline filter material, reflecting a flow state of the water after passing through the filter material. The water flow speed value for the alkaline filter material can be either the first water flow speed value or the second water flow speed value.

The temperature deviation severity can be obtained by calculating a difference or ratio between the temperature deviation degree and the preset temperature deviation degree threshold. The temperature deviation severity reflects a magnitude of the difference between the current temperature and the optimal measurement temperature.

An increase in water flow speed enhances a convective heat transfer coefficient, thereby affecting an efficiency of heat exchange between the water flow and its surrounding environment (such as water pipeline walls, other components in the water treatment equipment, etc.). This heat exchange affects a temperature distribution of the water flow, which in turn may affect an accuracy of temperature measurement. Therefore, when considering temperature correction, the impact of water flow speed on the temperature distribution and measurement must be taken into account. By combining the water flow speed value and the temperature deviation severity, the correction adjustment coefficient can be calculated, and the preset temperature correction coefficient can be adjusted based on the calculated correction adjustment coefficient.

The correction adjustment coefficient can be calculated using the following formula:

Kf=α·RV+β·δT/T0;

Wherein Kf represents the correction adjustment coefficient, α and β are weighting coefficients used to adjust an impact degree of the water flow speed value (RV) and the temperature deviation severity (ΔT/T0) on the temperature correction coefficient, ΔT represents the temperature deviation degree, and T0 represents the preset temperature deviation degree threshold.

ΔT/T0 represents the relative magnitude of the temperature deviation. This value is a dimensionless ratio used to measure the temperature deviation severity.

A combination of α·RV+β·ΔT/T0 takes into account the impact of the water flow speed value and the temperature deviation degree on the temperature correction coefficient. By adjusting the values of α and β, the impact degree of these two factors on the correction adjustment coefficient can be changed.

The aforementioned optional embodiment, helps to more accurately evaluate the impact of water flow state on the temperature measurement by obtaining the water flow speed value, thereby enabling more reasonable decisions in adjusting the temperature correction coefficient. The calculation of the temperature deviation severity helps a more precise understanding of the impact of the current environmental on the temperature measurement, providing more targeted guidance for subsequent adjustments to the temperature correction coefficient.

In block S34, calculating and obtaining a pH value of water based on the calculated target difference value in dissolved substance content and the target water flow temperature value.

According to a first target mapping relationship, the pH value of water can be calculated based on the target water flow temperature value and the target difference value in dissolved substance content. The first target mapping relationship is used to represent correspondences between the target water flow temperature values, the difference values in dissolved substance content, and the pH values of the water.

To obtain the first target mapping relationship, it is necessary to collect water quality sample data from different water sources and at various water flow temperatures in advance. Based on the water quality sample data, a potential correlation or functional relationship between the target water flow temperature values, the difference values in dissolved substance content, and the pH values can be fitted. Using the fitted correlation or functional relationship, the pH value of water can then be calculated based on the target water flow temperature value and the target difference value in dissolved substance content.

The water quality sample data can include target water flow temperature values, difference values in dissolved substance content, and corresponding pH values. Collecting the water quality sample data from different water sources and at various water flow temperatures is to ensure that the functional relationship fitted based on the water quality sample data has sufficient generalization ability. Water quality conditions are dynamically changing and impacted by various factors. By collecting diversified water quality sample data, it can be ensured that the model can learn complex relationships between different water quality characteristics, in order to better adapt to trends of water quality parameters changing over time and space, and thus providing more reliable prediction results.

The collected water quality sample data can be preprocessed, including removing outliers and smoothing data, etc., to improve data quality.

An appropriate data fitting algorithm (such as a linear regression, a polynomial regression, a machine learning algorithm, etc.) can be used to fit the functional relationship between the target water flow temperature values, the difference values in dissolved substance content (e.g., ΔTDS), and the pH values, in order to obtain a mathematical function that best describes the relationship between them. For example, by linearly fitting target water flow temperature values T, difference values in dissolved substance content ΔTDS, and pH values, a following relationship equation can be obtained: pH=a+bΔTDS+cT. Wherein, a is a source term constant, b is an amplitude index, and c is a temperature correction coefficient. All three are related to the alkaline properties of mineralized filter material and are measured experimentally.

As the temperature increases, the ionization degree of water will increase, resulting in an increase in the number of hydrogen ions and hydroxide ions ionized. Simultaneously, it will affect the solubility of minerals and salts dissolved in the water. Some minerals and salts have increased solubility when water temperature rises, but decreased solubility when water temperature drops. Therefore, differences in upstream water flow temperatures and downstream water flow temperatures may lead to changes in the amount of minerals and salts dissolved in the water, thereby affecting the upstream and downstream dissolved substance content (TDS values or conductivity).

In the embodiments of the present disclosure, obtaining the target water flow temperature value in the water treatment equipment includes acquiring a first target water flow temperature value upstream of the alkaline filter material and a second target water flow temperature value downstream of the alkaline filter material.

A first temperature sensor can be installed upstream of the alkaline filter material. The first temperature sensor is configured to detect a real-time water flow temperature value upstream of the alkaline filter material and to correct the detected real-time water flow temperature value to obtain the first target water flow temperature value. Similarly, a second temperature sensor can be installed downstream of the alkaline filter material. The second temperature sensor is configured to detect a real-time water flow temperature value downstream of the alkaline filter material and to correct the detected real-time water flow temperature value to obtain the second target water flow temperature value.

In an optional embodiment, the step of calculating and obtaining a pH value of water based on the calculated target difference value in dissolved substance content and the target water flow temperature value, includes the following:
calculating the pH value of water based on the calculated target difference value in dissolved substance content, the first target water flow temperature value, and the second target water flow temperature value.

According to a second target mapping relationship, the pH value of water can be calculated based on the first target water flow temperature value, the second target water flow temperature value, and the target difference value in dissolved substance content. The second target mapping relationship is used to represent correspondences between the first target water flow temperature values, the second target water flow temperature values, the difference values in dissolved substance content, and the pH values of the water.

To obtain the first target mapping relationship, it is necessary to collect water quality sample data from different water sources and conditions in advance. Based on the water quality sample data, a potential correlation or functional relationship between the first target water flow temperature values, the second target water flow temperature values, the difference values in dissolved substance content, and the pH values can be fitted. Using the fitted correlation or functional relationship, the pH value of water can then be calculated based on the first target water flow temperature value, the second target water flow temperature value and the target difference value in dissolved substance content.

The water quality sample data can include the first target water flow temperature values, the second target water flow temperature values, the difference values in dissolved substance content, and corresponding pH values. Collecting the water quality sample data from different water sources and conditions is to ensure that the functional relationship fitted based on the water quality sample data has sufficient generalization ability. Water quality conditions are dynamically changing and impacted by various factors. By collecting diversified water quality sample data, it can be ensured that the model can learn complex relationships between different water quality characteristics, in order to better adapt to trends of water quality parameters changing over time and space, and thus providing more reliable prediction results.

The collected water quality sample data can be preprocessed, including removing outliers and smoothing data, etc., to improve data quality.

An appropriate data fitting algorithm (such as a linear regression, a polynomial regression, a machine learning algorithm, etc.) can be used to fit the functional relationship between the first target water flow temperature values, the second target water flow temperature values, the difference values in dissolved substance content (e.g., ΔTDS), and the pH values, in order to obtain a mathematical function that best describes the relationship between them.

The above optional embodiment, if only considering the target difference value in dissolved substance content and the first target water flow temperature value, may result in errors due to neglecting temperature changes. By introducing the second target water flow temperature value, this error can be better corrected and the impact of temperature changes on pH value throughout the entire water treatment process can be more comprehensively taken into account. This helps to more accurately reflect water quality conditions and improves the accuracy of calculation results. In addition, introducing the second target water flow temperature value when calculating the pH value of water can achieve more real-time detection and warning functions. When there is an abnormal change in water flow temperature, problems can be detected in a timely manner and corresponding measures can be taken to ensure water quality safety.

In another optional embodiment, the step of calculating and obtaining a pH value of water based on the calculated target difference value in dissolved substance content and the target water flow temperature value, includes the following:
calculating a difference value in water flow temperature based on the first and the second target water flow temperature value;
calculating the pH value of water based on the calculated target difference value in dissolved substance content and the difference value in water flow temperature.

According to a third target mapping relationship, the pH value of water can be calculated based on the difference value in water flow temperature, and the target difference value in dissolved substance content. The third target mapping relationship is used to represent correspondences between the difference value in water flow temperature, the difference values in dissolved substance content, and the pH values of the water.

To obtain the third target mapping relationship, it is necessary to collect water quality sample data from different water sources and conditions in advance. Based on the water quality sample data, a potential correlation or functional relationship between the difference values in water flow temperature, the difference values in dissolved substance content, and the pH values can be fitted. Using the fitted correlation or functional relationship, the pH value of water can then be calculated based on the difference value in water flow temperature and the target difference value in dissolved substance content.

The water quality sample data can include the difference values in water flow temperature, the difference values in dissolved substance content, and corresponding pH values. Collecting the water quality sample data from different water sources and conditions is to ensure that the functional relationship fitted based on the water quality sample data has sufficient generalization ability. Water quality conditions are dynamically changing and impacted by various factors. By collecting diversified water quality sample data, it can be ensured that the model can learn complex relationships between different water quality characteristics, in order to better adapt to trends of water quality parameters changing over time and space, and thus providing more reliable prediction results.

The collected water quality sample data can be preprocessed, including removing outliers and smoothing data, etc., to improve data quality.

An appropriate data fitting algorithm (such as a linear regression, a polynomial regression, a machine learning algorithm, etc.) can be used to fit the functional relationship between the difference values in water flow temperature, the difference values in dissolved substance content (e.g., ΔTDS), and the pH values, in order to obtain a mathematical function that best describes the relationship between them.

In summary, the methods for obtaining the pH value of water provided in the present disclosure include the following three types.

Scheme One: calculating and obtaining the pH value of water based on the calculated target difference value in dissolved substance content and the first target water flow temperature value.

Scheme Two: calculating and obtaining the pH value of water based on the calculated target difference value in dissolved substance content, the first target water flow temperature value, and the second target water flow temperature value.

Scheme Three: calculating and obtaining the pH value of water based on the calculated target difference value in dissolved substance content and difference value in water flow temperature (the difference value between the first target water flow temperature value and the second target water flow temperature value).

The above Scheme One only considers the target difference value in dissolved substance content and the first target water flow temperature value, while ignoring the temperature changes of the water flow during the treatment process, which may affect the accuracy of the pH value. The above Scheme Three, comprehensively takes into account the temperature changes of the water flow throughout the entire treatment process, by introducing the difference value in water flow temperature, thereby improving the accuracy of the pH value calculation.

Although the above Scheme Two considers both the first target water flow temperature value and the second target water flow temperature value, it does not explicitly use the difference value between the two to directly reflect the temperature changes of the water flow. The above Scheme Three, however, explicitly uses the difference value in water flow temperature, which more directly reflects the temperature changes of the water flow during the treatment process, thus improving the accuracy of the pH value calculation. Furthermore, in Scheme Two, both the first target water flow temperature value and the second target water flow temperature value need to be considered simultaneously, and a complex model containing two temperature variables is required to established to calculate the pH value. In contrast, in Scheme Three, since the difference value in water flow temperature has already been calculated, the calculation process can be simplified, and only a model containing the difference value in water flow temperature and the target difference value in dissolved substance content is established. Using the difference value in water flow temperature as an input variable for the model makes the model easier to understand and interpret. This is because the difference value in water flow temperature directly reflects the temperature changes of the water flow during the treatment process, and the impact of this change on the pH value is obvious.

The water quality pH detection method provided in the embodiments of the present disclosure directly obtains the first dissolved substance content value upstream and the second dissolved substance content value downstream of the alkaline filter material in the water treatment equipment, providing a direct basis for subsequent pH value calculations. Based on the second dissolved substance content value and the first dissolved substance content value, the target difference value in dissolved substance content is calculated. The target difference value in dissolved substance content represents changes in dissolved substance content of the water during the treatment process and can be used to detect changes in water quality. Since the temperature of water affects its ionization degree and the activity of dissolved substances, the pH value will be affected. by comprehensively considering the two key factors of water flow temperature and the target difference value in dissolved substance content, the present disclosure can more accurately reflect the changes in water quality, and improve the accuracy and reliability of pH value calculations. Additionally, the present disclosure avoids a use of expensive pH meters, and reduces a cost of water quality detection.

By detecting the water quality pH value in real-time, problems in the operation of the water treatment equipment, such as aging of filter material and insufficient chemical reactions, can be detected in a timely manner, allowing for timely adjustments and optimizations, thereby improving the operational efficiency and service life of the equipment.

An embodiment of the present disclosure also provides a computer-readable storage medium having stored thereon computer programs, which, when executed by a processor, performs the following steps:
acquiring a first dissolved substance content value upstream of an alkaline filter material and a second dissolved substance content value downstream of the alkaline filter material in water treatment equipment;
calculating a target difference value in dissolved substance content based on the first and the second dissolved substance content values;
calculating and obtaining a pH value of water based on the calculated target difference value in dissolved substance content.

In an optional embodiment, when executed by the processor, the computer programs implement the step of calculating a target difference value in dissolved substance content based on the first and the second dissolved substance content values, which specifically includes:
acquiring a first water flow speed upstream of the alkaline filter material and a second water flow speed downstream of the alkaline filter material;
correcting the first dissolved substance content value using the first water flow speed to obtain a first corrected dissolved substance content value;
correcting the second dissolved substance content value using the second water flow speed to obtain a second corrected dissolved substance content value;
obtaining the target difference value in dissolved substance content based on the first and the second corrected dissolved substance content values.

In an optional embodiment, when executed by the processor, the computer programs implement the step of correcting the first dissolved substance content value using the first water flow speed to obtain a first corrected dissolved substance content value, which specifically includes:
determining a change in water flow speed based on the first water flow speed and a first water flow speed of a previous cycle;
correcting the first dissolved substance content value based on the first water flow speed and the change in water flow speed through a preset first correction model, and obtaining the first corrected dissolved substance content value.

In an optional embodiment, when executed by the processor, the computer programs implement the step of correcting the second dissolved substance content value using the second water flow speed to obtain a second corrected dissolved substance content value, which specifically includes:
calculating a water residence time based on a volume of the alkaline filter material and the second water flow speed;
determining a mass transfer coefficient based on the second water flow speed;
correcting the second dissolved substance content value based on a preset reference water flow speed, the water residence time, the mass transfer coefficient, and the second water flow speed through a preset second correction model, and obtaining the second corrected dissolved substance content value.

In an optional embodiment, when executed by the processor, the computer programs implement the step of calculating and obtaining a pH value of water based on the calculated target difference value in dissolved substance content, which specifically includes:
determining a type of the alkaline filter material; selecting a first target water quality pH detection model from a plurality of preset first water quality pH detection models based on the type; and calculating and obtaining the pH value of water through the first target water quality pH detection model based on the calculated target difference value in dissolved substance content; or
determining a location of the water treatment equipment; selecting a second target water quality pH detection model from a plurality of preset second water quality pH detection models based on the location; and calculating and obtaining the pH value of water through the second target water quality pH detection model based on the calculated target difference value in dissolved substance content; or
determining a type of the alkaline filter material and a location of the water treatment equipment; selecting a third target water quality pH detection model from a plurality of preset third water quality pH detection models based on the type and the location; and calculating and obtaining the pH value of water through the third target water quality pH detection model based on the calculated target difference value in dissolved substance content.

In an optional embodiment, when executed by the processor, the computer programs further implement the following steps:

obtaining a target water flow temperature value in the water treatment equipment.

Accordingly, when executed by the processor, the computer programs implement the step of calculating and obtaining a pH value of water based on the calculated target difference value in dissolved substance content, which specifically includes: calculating and obtaining a pH value of water based on the calculated target difference value in dissolved substance content and the target water flow temperature value.

In an optional embodiment, when executed by the processor, the computer programs implement the step of obtaining a target water flow temperature value in the water treatment equipment, which specifically includes:
acquiring a real-time water flow temperature value in the water treatment equipment;
comparing the real-time water flow temperature value with a preset reference temperature range;
when the real-time water flow temperature value is within the preset reference temperature range, correcting the real-time water flow temperature value using a preset temperature correction coefficient to obtain the target water flow temperature value;
when the real-time water flow temperature value is not within the preset reference temperature range, adjusting the preset temperature correction coefficient, and correcting the real-time water flow temperature value using the adjusted temperature correction coefficient to obtain the target water flow temperature value.

In an optional embodiment, when executed by the processor, the computer programs implement the step of adjusting the preset temperature correction coefficient, which specifically includes:
obtaining a temperature deviation degree based on the real-time water flow temperature value and the preset reference temperature range;
adjusting the preset temperature correction coefficient according to the temperature deviation degree.

In an optional embodiment, when executed by the processor, the computer programs implement the step of adjusting the preset temperature correction coefficient according to the temperature deviation degree, which specifically includes:
obtaining a temperature deviation severity based on the temperature deviation degree and a preset temperature deviation degree threshold;
determining a water flow speed value of the alkaline filter material;
obtaining a correction adjustment coefficient based on the water flow speed value and the temperature deviation severity;
adjusting the preset temperature correction coefficient based on the correction adjustment coefficient.

In an optional embodiment, when executed by the processor, the computer programs implement the step of obtaining a target water flow temperature value, which specifically includes:
acquiring a first target water flow temperature value upstream of the alkaline filter material and acquiring a second target water flow temperature value downstream of the alkaline filter material.

Accordingly, when executed by the processor, the computer programs implement the step of calculating and obtaining a pH value of water based on the calculated target difference value in dissolved substance content and the target water flow temperature value, which specifically includes:
calculating the pH value of water based on the calculated target difference value in dissolved substance content, the first target water flow temperature value, and the second target water flow temperature value.

In an optional embodiment, when executed by the processor, the computer programs implement the step of obtaining a target water flow temperature value, which specifically includes:
acquiring a first target water flow temperature value upstream of the alkaline filter material and acquiring a second target water flow temperature value downstream of the alkaline filter material.

Accordingly, when executed by the processor, the computer programs implement the step of calculating and obtaining a pH value of water based on the calculated target difference value in dissolved substance content and the target water flow temperature value, which specifically includes:
calculating a difference value in water flow temperature based on the first and the second target water flow temperature value;
calculating the pH value of water based on the calculated target difference value in dissolved substance content and the difference value in water flow temperature.

In an optional embodiment, when executed by the processor, the computer programs further implement the following steps:
displaying the pH value of water using a preset display mode; and/or
comparing the pH value of water with a preset pH threshold range; and triggering an alarm according to a preset alarm mode, when the pH value of water exceeds the preset pH threshold range.

The present disclosure directly obtains the first dissolved substance content value upstream and the second dissolved substance content value downstream of the alkaline filter material in the water treatment equipment, providing a direct basis for subsequent pH value calculations. Based on the second dissolved substance content value and the first dissolved substance content value, the target difference value in dissolved substance content is calculated. The target difference value in dissolved substance content represents changes in dissolved substance content of during a water treatment process and can be used to detect changes in water quality. Finally, based on the calculated target difference value in dissolved substance content, the pH value of water is calculated, greatly improving an accuracy of water quality detection. In addition, the present disclosure avoids a use of expensive pH meters and reduces a cost of water quality detection.

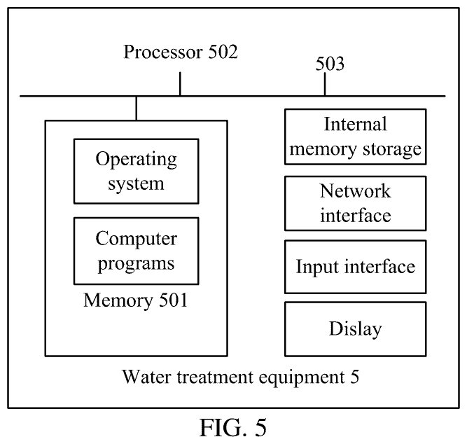

FIG. 5 shows a schematic structural diagram of water treatment equipment provided by an embodiment of the present disclosure. In a preferred embodiment of the present disclosure, the water treatment equipment 500 can include: a memory 501, at least one processor 502, at least one communication bus 503.

It should be understood by those skilled in the art that the structure of the water treatment equipment 500 shown in FIG. 5 does not constitute a limitation of the embodiment of the present disclosure. The water treatment equipment 500 may also include more or less hardware or software than illustrated, or may have different component arrangements, such as an internal memory storage, a network interface, a display.

In some embodiments, the water treatment equipment 500 can be a device that is capable of automatically performing numerical calculations and/or information processing in accordance with pre-set or stored instructions. It's hardware can include, but is not limited to, microprocessors, application specific integrated circuits (ASICs), programmable gate arrays (PGAs), digital signal processors (DSPs), and embedded systems. The water treatment equipment 500 may further include a customer device. The customer device includes but is not limited to any electronic product that can interact with a user through a keyboard, a mouse, a remote controller, a touch panel or a voice control device, for example, individual computers, tablets, smartphones, digital cameras, and so on.

It should be noted that the water treatment equipment 500 is merely an example, and other existing or future electronic products that may be suitable for the present disclosure should also be included in the scope of the present disclosure, and are hereby incorporated by reference.

In some embodiments, the memory 501 stores computer programs that, when executed by the at least one processor 502, implements all or part of the steps in the water quality pH detection method as described. The memory 501 also stores at least one operating system. The memory 501 can include a read-only memory (ROM), a programmable read-only memory (PROM), an erasable programmable read only memory (EPROM), an one-time programmable read-only memory (OTPROM), an electronically-erasable programmable read-only memory (EEPROM), a compact disc read-only memory (CD-ROM), or any other computer-readable medium capable of carrying or storing data. Furthermore, the computer-readable storage medium may mainly include a program storage area and a data storage area, wherein the program storage area may store an operating system, application programs required for at least one function, and so on.

In some embodiments, the at least one processor 502 is a control unit of the water treatment equipment 500, which connects various components of the water treatment equipment 500 using various interfaces and lines. By running or executing computer programs or modules stored in the memory 501, and by invoking the data stored in the memory 501, the at least one processor 502 can perform various functions of the water treatment equipment 500 and process data of the water treatment equipment 500. For example, when executing the computer programs stored in the memory, the at least one processor 502 implements all or part of the steps in the water quality pH detection method described in the embodiment of the present disclosure; or it implements all or part of the functions of a water quality pH detection device. The at least one processor 502 can be composed of integrated circuits, for instance, it can compose of a single packaged integrated circuit, or it can be composed of a plurality of integrated circuits packaged with the same or different functions, including combinations of one or more central processing units (CPUs), microprocessors, digital processing chips, graphics processors, and various control chips.

In an optional embodiment, when executing the computer programs stored in the memory, the at least one processor 502 implements all or part of the steps in the water quality pH detection method. Reference is made to FIG. 1 and/or FIG. 3 and their related descriptions for the water quality pH detection method. For the sake of brevity in the specification, this application will not elaborate on the specific steps of the water quality pH detection method here.

In some embodiments, the at least one communication bus 503 is configured to achieve connection and communication between the memory 501 and the at least one processor 502, and other components of the water treatment equipment 500. Although it is not shown, the water treatment equipment 500 may further include a power supply (such as a battery) for powering various components. Preferably, the power supply may be logically connected to the at least one processor 502 through a power management device, thereby, the power management device manages functions such as charging, discharging, and power management. The power supply can include one or more a DC or AC power source, a recharging device, a power failure detection circuit, a power converter or inverter, a power status indicator, and the like. The water treatment equipment 500 may further include various sensors, such as a BLUETOOTH module, a Wi-Fi module, and the like, and details are not described herein.

The above-described integrated unit implemented in a form of software function modules can be stored in a computer readable storage medium. The above software function modules are stored in a storage medium, and includes a plurality of instructions for causing water treatment equipment (which may be a personal computer, or a network device, etc.) or a processor to execute the method according to various embodiments of the present disclosure.

In several embodiments provided in the present disclosure, it should be understood that the disclosed device and method can be implemented in other ways. For example, the device embodiments described above are merely illustrative. For example, divisions of the module are only a logical function division, and there can be other division ways in actual implementation.

The modules described as separate components may or may not be physically separated, and the components displayed as modules may or may not be physical units. That is, it can locate in one place, or distribute to multiple network units. Some or all of the modules can be selected according to actual needs to achieve the purpose of the solution of above embodiments.

What is claimed is:

1. A water quality pH detection method applicable in water treatment equipment, the method comprising:
acquiring a first dissolved substance content value upstream of an alkaline filter material and a second dissolved substance content value downstream of the alkaline filter material in the water treatment equipment;
calculating a target difference value in dissolved substance content based on the first and the second dissolved substance content values; and
calculating and obtaining a pH value of water based on the calculated target difference value in dissolved substance content.

2. The water quality pH detection method according to claim 1, wherein the step of calculating a target difference value in dissolved substance content based on the first and the second dissolved substance content values comprises:
acquiring a first water flow speed upstream of the alkaline filter material and a second water flow speed downstream of the alkaline filter material;
correcting the first dissolved substance content value using the first water flow speed to obtain a first corrected dissolved substance content value;
correcting the second dissolved substance content value using the second water flow speed to obtain a second corrected dissolved substance content value; and
obtaining the target difference value in dissolved substance content based on the first and the second corrected dissolved substance content values.

3. The water quality pH detection method according to claim 2, wherein the step of correcting the first dissolved substance content value using the first water flow speed to obtain a first corrected dissolved substance content value comprises:
determining a change in water flow speed based on the first water flow speed and a first water flow speed of a previous cycle; and
correcting the first dissolved substance content value based on the first water flow speed and the change in water flow speed through a preset first correction model, and obtaining the first corrected dissolved substance content value.

4. The water quality pH detection method according to claim 3, wherein the step of correcting the second dissolved substance content value using the second water flow speed to obtain a second corrected dissolved substance content value comprises:
calculating a water residence time based on a volume of the alkaline filter material and the second water flow speed;
determining a mass transfer coefficient based on the second water flow speed; and
correcting the second dissolved substance content value based on a preset reference water flow speed, the water residence time, the mass transfer coefficient, and the second water flow speed through a preset second correction model, and obtaining the second corrected dissolved substance content value.

5. The water quality pH detection method according to claim 4, wherein the step of calculating and obtaining a pH value of water based on the calculated target difference value in dissolved substance content comprises:
determining a type of the alkaline filter material; selecting a first target water quality pH detection model from a plurality of preset first water quality pH detection models based on the type; and calculating and obtaining the pH value of water through the first target water quality pH detection model based on the calculated target difference value in dissolved substance content; or
determining a location of the water treatment equipment; selecting a second target water quality pH detection model from a plurality of preset second water quality pH detection models based on the location; and calculating and obtaining the pH value of water through the second target water quality pH detection model based on the calculated target difference value in dissolved substance content; or
determining a type of the alkaline filter material and a location of the water treatment equipment; selecting a third target water quality pH detection model from a plurality of preset third water quality pH detection models based on the type and the location; and calculating and obtaining the pH value of water through the third target water quality pH detection model based on the calculated target difference value in dissolved substance content.

6. The water quality pH detection method according to claim 1, further comprising:
obtaining a target water flow temperature value in the water treatment equipment;
wherein the step of calculating and obtaining a pH value of water based on the calculated target difference value in dissolved substance content comprises: calculating and obtaining a pH value of water based on the calculated target difference value in dissolved substance content and the target water flow temperature value.

7. The water quality pH detection method according to claim 6, wherein the step of obtaining a target water flow temperature value in the water treatment equipment comprises:
acquiring a real-time water flow temperature value in the water treatment equipment;
comparing the real-time water flow temperature value with a preset reference temperature range;
when the real-time water flow temperature value is within the preset reference temperature range, correcting the real-time water flow temperature value using a preset temperature correction coefficient to obtain the target water flow temperature value;
when the real-time water flow temperature value is not within the preset reference temperature range, adjusting the preset temperature correction coefficient, and correcting the real-time water flow temperature value using the adjusted temperature correction coefficient to obtain the target water flow temperature value.

8. The water quality pH detection method according to claim 7, wherein the step of adjusting the preset temperature correction coefficient comprises:
obtaining a temperature deviation degree based on the real-time water flow temperature value and the preset reference temperature range;
adjusting the preset temperature correction coefficient according to the temperature deviation degree.

9. The water quality pH detection method according to claim 8, wherein the step of adjusting the preset temperature correction coefficient according to the temperature deviation degree comprises:
obtaining a temperature deviation severity based on the temperature deviation degree and a preset temperature deviation degree threshold;
determining a water flow speed value of the alkaline filter material;
obtaining a correction adjustment coefficient based on the water flow speed value and the temperature deviation severity;
adjusting the preset temperature correction coefficient based on the correction adjustment coefficient.

10. The water quality pH detection method according to claim 9, wherein the step of obtaining a target water flow temperature value comprises:
acquiring a first target water flow temperature value upstream of the alkaline filter material and acquiring a second target water flow temperature value downstream of the alkaline filter material;
wherein the step of calculating and obtaining a pH value of water based on the calculated target difference value in dissolved substance content and the target water flow temperature value comprises: calculating the pH value of water based on the calculated target difference value in dissolved substance content, the first target water flow temperature value, and the second target water flow temperature value.

11. The water quality pH detection method according to claim 9, wherein the step of obtaining a target water flow temperature value, which specifically comprises:
acquiring a first target water flow temperature value upstream of the alkaline filter material and acquiring a second target water flow temperature value downstream of the alkaline filter material;
wherein the step of calculating and obtaining a pH value of water based on the calculated target difference value in dissolved substance content and the target water flow temperature value comprises: calculating a difference value in water flow temperature based on the first and the second target water flow temperature value; calculating the pH value of water based on the calculated target difference value in dissolved substance content and the difference value in water flow temperature.

12. The water quality pH detection method according to claim 1, further comprising:
displaying the pH value of water using a preset display mode; and/or comparing the pH value of water with a preset pH threshold range; and triggering an alarm according to a preset alarm mode, when the pH value of water exceeds the preset pH threshold range.

13. Water treatment equipment, comprising:
at least one processor; and
a memory storing computer programs, wherein the at least one processor, when executing the computer programs, implements the following steps:
acquiring a first dissolved substance content value upstream of an alkaline filter material and a second dissolved substance content value downstream of the alkaline filter material in the water treatment equipment;
calculating a target difference value in dissolved substance content based on the first and the second dissolved substance content values; and
calculating and obtaining a pH value of water based on the calculated target difference value in dissolved substance content.

14. The water treatment equipment according to claim 13, wherein the at least one processor, when executing the computer programs, implements the step of calculating a target difference value in dissolved substance content based on the first and the second dissolved substance content values, which comprises:
acquiring a first water flow speed upstream of the alkaline filter material and a second water flow speed downstream of the alkaline filter material;
correcting the first dissolved substance content value using the first water flow speed to obtain a first corrected dissolved substance content value;
correcting the second dissolved substance content value using the second water flow speed to obtain a second corrected dissolved substance content value;
obtaining the target difference value in dissolved substance content based on the first and the second corrected dissolved substance content values.

15. The water treatment equipment according to claim 13, wherein the at least one processor, when executing the computer programs, implements the following steps:
obtaining a target water flow temperature value in the water treatment equipment;
wherein the step of calculating and obtaining a pH value of water based on the calculated target difference value in dissolved substance content comprises: calculating and obtaining a pH value of water based on the calculated target difference value in dissolved substance content and the target water flow temperature value.

16. The water treatment equipment according to claim 15, wherein the at least one processor, when executing the computer programs, implements the steps of obtaining a target water flow temperature value in the water treatment equipment, which comprises:
acquiring a real-time water flow temperature value in the water treatment equipment;
comparing the real-time water flow temperature value with a preset reference temperature range;
when the real-time water flow temperature value is within the preset reference temperature range, correcting the real-time water flow temperature value using a preset temperature correction coefficient to obtain the target water flow temperature value;
when the real-time water flow temperature value is not within the preset reference temperature range, adjusting the preset temperature correction coefficient, and correcting the real-time water flow temperature value using the adjusted temperature correction coefficient to obtain the target water flow temperature value.

17. The water treatment equipment according to claim 13, wherein the at least one processor, when executing the computer programs, implements the following steps:
displaying the pH value of water using a preset display mode; and/or comparing the pH value of water with a preset pH threshold range; and triggering an alarm according to a preset alarm mode, when the pH value of water exceeds the preset pH threshold range.

18. A non-transitory storage medium having stored thereon instructions that, when executed by a processor of water treatment equipment, causes the processor to perform a water quality pH detection method, the method comprising:
acquiring a first dissolved substance content value upstream of an alkaline filter material and a second dissolved substance content value downstream of the alkaline filter material in water treatment equipment;
calculating a target difference value in dissolved substance content based on the first and the second dissolved substance content values; and
calculating and obtaining a pH value of water based on the calculated target difference value in dissolved substance content.

19. The non-transitory storage medium of claim 18, wherein when executed by the processor, the computer programs implement the step of calculating a target difference value in dissolved substance content based on the first and the second dissolved substance content values, which comprises:
acquiring a first water flow speed upstream of the alkaline filter material and a second water flow speed downstream of the alkaline filter material;
correcting the first dissolved substance content value using the first water flow speed to obtain a first corrected dissolved substance content value;
correcting the second dissolved substance content value using the second water flow speed to obtain a second corrected dissolved substance content value;
obtaining the target difference value in dissolved substance content based on the first and the second corrected dissolved substance content values.

20. The non-transitory storage medium of claim 18, wherein when executed by the processor, the computer programs further implement the steps:
obtaining a target water flow temperature value in the water treatment equipment;
wherein the step of calculating and obtaining a pH value of water based on the calculated target difference value in dissolved substance content comprises: calculating and obtaining a pH value of water based on the calculated target difference value in dissolved substance content and the target water flow temperature value.
